(19) United States
(12) Patent Application Publication
(10) Pub. No.: US 2022/0354996 A1
(43) Pub. Date: Nov. 10, 2022

(54) SURGICAL MEMBRANE

(71) Applicant: NEOSS LIMITED, Yorkshire (GB)

(72) Inventors: Fredrik Nils ENGMAN, Goteborg (SE); Herman SAHLIN, Goteborg (SE); My HEDHAMMAR, Stockholm (SE); Sarunas PETRONIS, Partille (SE); Christos Panagiotis TASIOPOULOS, Solna (SE)

(21) Appl. No.: 17/770,582

(22) PCT Filed: Oct. 22, 2020
(86) PCT No.: PCT/GB2020/052666

§ 371 Date: Apr. 20, 2022

(30) Foreign Application Priority Data
Oct. 23, 2019 (GB) 1915336.0

(51) Int. Cl. A61L 27/54; A61L 27/36; A61L 27/28

(52) U.S. Cl. CPC A61L 27/54; A61L 27/3604; A61L 27/3687; A61L 27/28; A61L 2300/412; A61L 2430/02; A61L 2430/12; A61L 2420/02

(57) ABSTRACT

A surgical membrane for supporting bone growth comprises a surface configured for receiving a surface functionalisation agent capable of promoting cell adhesion and proliferation and/or of reducing bacterial growth on said surface. The membrane is also subjected to a treatment improving the wettability of the surface.

### BACKGROUND

It is known to use PTFE based membranes during surgical procedures, in particular in dental surgery, due to their excellent mechanical properties and exceptional biocompatibility. Being non-resorbable and chemically inert, the PTFE membranes are widely used in the fields of dental and bone surgeries. The membrane generally acts as a barrier to prevent rapidly migrating connective tissue cells from entering a bone defect so that slower migrating cells with osteogenic potential can preferentially enter the bone defect and assist with bone growth. A non-resorbable membrane is removed after sufficient bone growth has been achieved which, depending on situation and clinical parameters, generally takes between 1-6 months. Early PTFE membranes featured an open structure that allowed extensive tissue ingrowth, which could complicate the retrieval procedures and lead to the bacteria populating the membrane material itself and/or penetrating the material, thus requiring early surgical intervention. This led to a new generation of membranes with a much denser, almost or completely solid, material in order to improve retrieval and bacteria penetration. However, these dense membranes became known in the art as being poor cell adhesion promoters resulting in for example compromised stability during function, potentially due to their surface hydrophobicity. This surface property discourages cells to adhere to the membrane post-surgery, thus slowing down wound healing and, in turn, increasing the risk of bacterial infection.

Microorganisms, particularly bacteria, can become entrapped in a matrix of the PTFE membrane, with consequential forming of a biofilm, thus leading to the post-operative infection that can spread into the surrounding body tissues and be further transported by bodily fluids, for example blood, to the other body organs, urinary tract, and even bones. This will require treatment with antibiotics of both the surgical site by using topical antiseptics and administering drugs orally and/or intravenously. Mistreatment of the surgical wound infection can lead to a secondary infection which, coupled with the slow tissue regrowth around the implant site, can be debilitating for a patient.

Hence, it is desirable to improve osseointegration, cell proliferation and to reduce the possibility of bacterial infection at the implant site.

It is an object of embodiments of the invention to at least mitigate one or more of the problems associated with the prior art.

### BRIEF SUMMARY OF THE DISCLOSURE

Aspects and embodiments of the invention provide a surgical membrane and a method of manufacturing thereof, as claimed in the appended claims.

In accordance with the present invention there is provided a surgical membrane for supporting bone growth, the membrane having a surface configured for receiving a surface functionalisation agent capable of promoting cell adhesion and proliferation and/or of reducing bacterial growth on said surface, the membrane also having been subjected to a treatment improving the wettability of the surface. Advantageously, the surgical membrane is provided with variable surface topography and chemical composition, the membrane is configured to accelerate the wound healing process and to mitigate bacterial invasion/spreading. That is, cell proliferation is affected by the wettability and the surface topography of the membrane surface, with the roughened and hydrophilic surface being more favourable for cell adhesion. Cellular morphology and bioactivity also change when the surface of the membrane changes from smooth to rough, therefore it is important to provide a membrane that will positively affect the tissue response during various bone formation stages.

In an embodiment, the treatment comprises chemical etching, ion bombardment, discharge plasma or UV-ozone treatment. Advantageously, this approach allows to modify surface in a reproducible and economic manner, with easy and repeatable control of the process parameters.

In another embodiment the treatment comprises using a polar solvent to lower surface tension and/or improve wettability of the surface. Optionally, the polar solvent comprises ethanol, methanol, propanol, isopropanol, or a mixture thereof. Optionally, the treatment further comprises gradually replacing the solvent with water. Advantageously, this provides a pre-bonding treatment that will allow to alter the surface affinity to the hydrophilic compounds, thus making the membrane surface susceptible to coating with hydrophilic agents and/or to improving cells adhesion to the said surface. Furthermore, treating the membrane surface with alcohols, with some of them widely used as antiseptic agents, allows to prevent bacterial contamination of the membrane pre-surgery.

In an embodiment, the surface functionalisation agent comprises a synthetic or biotechnologically produced material. Optionally, said material is recombinant spider silk protein. Optionally, the recombinant spider silk protein is native or modified with bioactive peptides. Optionally, the surface functionalisation agent is self-assembled into a nanofibrillar coating. Advantageously, coating of the membrane surface with fibrillar structure allows to create a unique strand-like network that will allow better cell adherence and, in turn, improve cell viability and promote tissue growth.

In an embodiment, at least a part of the membrane is non-resorbable. Advantageously, this provides a stable, non-degradable and biocompatible barrier that provides support and is resistant to breakdown by host tissues.

In an embodiment, the membrane comprises a polymer. Optionally, the surgical membrane comprises multidirectional PTFE, monodirectional PTFE, or a combination thereof. Advantageously, the use of different types of PTFE provides a variety of mechanical and morphological properties (tensile strength, creep, cold flow resistance, density, porosity) of the membrane, thus making the membrane suitable for a variety of medical applications.

In an embodiment, at least a part of the surgical membrane is resorbable, i.e. capable of breaking down and be absorbed by the surrounding tissues. This is advantageous when the membrane does not need to be removed, hence a second surgical intervention is avoided. Rapid resorption is also beneficial when there is a risk of bacterial infection, and the membrane is resorbed at the early stages, thus preventing bacterial growth.

In an embodiment, the surface is hydrophobic before being subjected to said treatment.

In yet another embodiment, the surface comprises a surface geometry, detectable at the micron or submicron level, which is capable of retaining said surface functionalisation agent. This is advantageous, because attachment of the surface functionalisation agent provides anchor points for the surrounding tissue cells to adhere and thus facilitating cellular osteodifferentiation and, in turn, promoting tissue ingrowth.

In an embodiment, surface geometry comprises at least one blind hole and/or a plurality of pores. Said holes and/or pores comprises a pharmaceutically active substance. Loading an active pharmaceutical ingredient (API) into the openings of the membrane is advantageous as such a surface arrangement allows for controlled drug release of the API into the surrounding tissue, thus combating diseases at the implantation site.

In yet another embodiment, said surface geometry comprises a roughened surface. Altering surface topology exhibits preferable advantages in terms of promoting biological tissue response and improving healing times by affecting the process and rate of the osseointegration of the surgical implant.

### BRIEF DESCRIPTION OF THE DRAWINGS

One or more embodiments of the invention will now be described by way of example only, with reference to the accompanying drawings, in which:

FIG. 1 shows a non-treated surgical membrane according to an embodiment of the invention; and

FIG. 2 shows a treated surgical membrane according to another embodiment of the invention.

### DETAILED DESCRIPTION

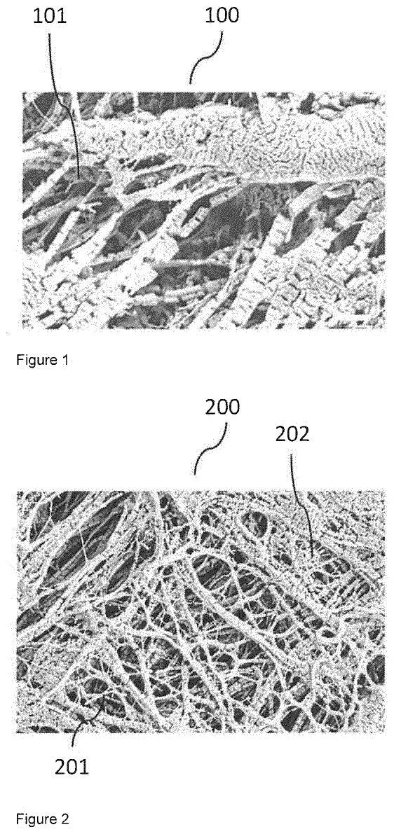

FIG. 1 shows a surgical membrane 100 according to an embodiment of the invention.

It comprises a layer in the form of a PTFE material. Optionally, the membrane comprises multiple layers (not shown) with different surface roughness and/or porosity, which enhances bone augmentation and osteointegration of the membrane post-surgery. Multiple layers can be bonded to one another by any suitable bonding means. The membrane has a plurality of interconnected openings (open pores) 101 formed by the interwoven fibres of the membrane. It is also anticipated that the membrane may comprise other surface arrangements, like surface indentations resulting in a semi-open porous structure (known as rough surface).

Said open pores and/or surface indentations may be at a micron level or nano level. In the other words, the average size of a pore or of an impression on the surface lies in the range of 0.1 nm to 1000 microns. In alternative embodiments, the membrane may have larger indentations in a millimetre range, for example the average size of the pore and/or indentation can be in the range of 1-10 mm.

Said pores and/or indentations are formed by any suitable method, including but not limited to, stretching of the material, embossing (direct or indirect), chemical etching, ion bombardment, or discharge plasma.

The surface indentations can be in the form of a blind hole capable of containing a pharmaceutically active substance suitable for a controlled drug release. Said pharmaceutically active substances may include, without limitation, antimicrobial agents, bone healing accelerators, non-steroid anti-inflammatories and the like.

The membrane (or at least one layer of a multi-layered membrane) may be formed from a polymer, however metal membranes and/or layers can also be provided. Said polymer can comprise PTFE, wherein PTFE can be dense monodirectional PTFE or less dense expanded multidirectional PTFE. Said metal can also comprise titanium or titanium alloy, however other metals or metal alloys suitable for the use in the human or animal body are also considered.

The membrane can be of a flat configuration or a non-planar configuration pre-formed to various shapes in accordance with the membrane application within the body (i.e. to be attached for example to the tibial bone for bone reconstruction surgery or to a jaw bone during a dental implantation operation).

FIG. 2 shows a surgical membrane 200 with a treated surface. It can be seen that additional surface features 201 in the form of thin strands, as well as additional pores 202 caused by the treatment, are visible. The aim of the surface treatment is to lower surface tension of the membrane and to allow a surface functionalisation agent to self-assemble into a fibrillar coating to facilitate cell adherence.

The basic steps of the treatment include i) altering the membrane morphology in order to promote the functionalisation agent to adhere to the surface, and ii) further subjecting the treated membrane to the surface functionalisation agent that will promote cell attachment and sustain bone growth.

Step (i) can without limitation include several strategies aimed at lowering the surface tension, with one of them being altering the surface morphology using ion bombardment, UV/ozone light irradiation or plasma discharge, and with the other being mild treatment of a non-polar hydrophobic membrane surface with a polar solvent to alter wettability of the membrane. Non-chemical surface treatments are advantageous when a controlled roughness is required to be created on the membrane surface, whereas mild chemical treatment with solvents like ethanol, methanol, propanol, isopropanol or the combinations thereof changes the surface from being hydrophobic to becoming hydrophilic, thus allowing consecutive attachment of the surface functionalisation agents. Chemical etching can also be used to create additional indentations on the surface of the membrane. It is understood that both chemical and non-chemical strategies affect the surface topography and alter the surface tension, thus providing an improved adhering of the cells onto the membrane.

Step (ii) comprises the subsequent treatment of the membrane of step (i) with a surface functionalisation agent. During this step the agent is preferably but not necessarily self-assembled into a fibrillar semi complete or complete coating that enhances in vivo cell adhesion by creating a microenvironment, wherein the cells are provided with attachment points and can more easily adhere to the membrane's surface, thus resulting in an improved cell proliferation. Said functionalisation agents can without limitation include silk, recombinant spider silk, silk modified with bioactive peptides, silk protein(s) or a combination thereof.

It is also understood that it is possible to apply the process described as step (ii) to an untreated membrane surface.

Furthermore, this method is not limited to PTFE membranes and can be equally applied to other suitable polymeric and/or metal membranes. In the other words, a metal or a metal/polymer composite surgical membrane can be treated with the chemical and/or non-chemical methods described therein, and further treated with surface functionalisation agent to improve biological response.

The method of treating a surgical membrane is provided in Example 1. The example below should not be considered to be a limit on the scope of the appended claims.

### Example 1

Step 1. PTFE membranes (Neoss, Harrogate, UK) are submerged in 70% ethanol, sonicated (Branson 3510, Marshall Scientific, Hampton, N.H., USA) for 15 minutes, and incubated overnight in 70% ethanol. The next day, they are serially hydrated in 40 and 20% ethanol-water solutions for 10 minutes each step. The membranes were then submerged in sterile Milli-Q water, sonicated for 10 minutes, incubated for 10 minutes and finally, incubated with sterile phosphate-buffered saline (PBS) for 10 minutes before coating with the silk protein.

Step 2. The stock solution of 3.0 mg/mL of FN-4RepCT protein in PBS (Spiber Technologies, Stockholm, Sweden) is thawed at room temperature and spun down for a minute using a bench-top centrifuge. The protein is then diluted in PBS to a final concentration of 0.1 mg/mL, spun down for another minute, and finally added to the respective membranes. After 1 hour of incubation, the protein solution is removed and coated membranes are washed twice with PBS.

Throughout the description and claims of this specification, the words “comprise” and “contain” and variations of them mean “including but not limited to”, and they are not intended to (and do not) exclude other additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A surgical membrane for supporting bone growth, the membrane having a surface configured for receiving a surface functionalisation agent capable of promoting cell adhesion and proliferation and/or of reducing bacterial growth on said surface, the membrane also having been subjected to a treatment improving the receipt of the functionalisation agent by the surface through increased wettability.

2. The surgical membrane of claim 1, wherein the treatment comprises chemical etching, ion bombardment, discharge plasma, stretching of the material or embossing.

3. The surgical membrane of claim 1, wherein the treatment comprises using a polar solvent to lower surface tension and/or improve wettability of the surface.

4. The surgical membrane of claim 3, wherein the treatment further comprises gradually replacing the solvent with water.

5. The surgical membrane of claim 3, wherein said polar solvent comprises ethanol, methanol, propanol, isopropanol, or a mixture thereof.

6. The surgical membrane of claim 1, wherein the surface functionalisation agent comprises a synthetic or biotechnologically produced material.

7. The surgical membrane of claim 6, wherein the material is a recombinant spider silk protein.

8. The surgical membrane of claim 7, wherein the recombinant spider silk protein is native or modified with bioactive peptides.

9. The surgical membrane of claim 1, wherein the surface functionalisation agent is self-assembled into a nanofibrillar coating.

10. The surgical membrane of claim 1 wherein at least a part of the membrane is non-resorbable.

11. The surgical membrane of claim 10 wherein the membrane comprises a polymer.

12. The surgical membrane of claim 11 wherein the membrane comprises multidirectional PTFE, monodirectional PTFE, or a combination thereof.

13. The surgical membrane of claim 1, wherein at least a part of the membrane is resorbable.

14. The surgical membrane of claim 1, wherein the surface is hydrophobic before being subjected to said treatment.

15. The surgical membrane of claim 1, wherein the surface comprises a surface geometry, detectable at the micron or submicron level, which is capable of retaining said surface functionalisation agent.

16. The surgical membrane of claim 15 wherein said surface geometry comprises at least one blind hole.

17. The surgical membrane of claim 15 wherein said surface geometry comprises a plurality of pores.

18. The surgical membrane of claim 15 wherein the plurality of pores or the at least one blind hole comprises a pharmaceutically active substance.

19. The surgical membrane of any of, wherein said surface geometry comprises a roughened surface.

20. A method of manufacturing the surgical membrane of claim 1, the method comprising the steps of:
forming the surgical membrane with a surface configured for receiving a surface functionalisation agent;
treating the surface of the membrane to improve the receipt of the functionalisation agent by the surface through increased wettability;
applying said surface functionalisation agent to the surface of the membrane so as to promote cell adhesion and proliferation and/or to reduce bacterial growth on said surface.
